SEQUENCE LISTING (3 sequences)

SEQ ID NO: 1 - protein, 355 aa, Homo sapien
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 2 - DNA, 1065 bp, Homo sapien
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagc

SEQ ID NO: 3 - DNA, 6405 bp, Mus musculus
ttggggtgggggagcgggggacggatgagccaagggaggagacaagctgctgttagccagcttgctcagatgggcagccccaaggacggcctctcccacctccttgtgtgctcctgcctcctcgaagcctttcttggggtctccacggggatcaggaggcttcggttgttatatgctgccattgacccatcacatggctcctgtgtctgttgattaccaacaggcagaatggtcagatagtgcctcccatctctagatggccacggaagcagggctcttcacctctctccttgcatctctgggcacctctgggtaacacgtggaactcgcccagttctgtggacctaaccctatgctctgtgatattccgggaggtgcactccagaaactaacccaggaaatgttggcagggggcccagaggtctttcctctgcccaagaaccccagttttccattcattctctcctgtagccagagtcaggcagaccctggatgacagtcacaaaggttagggtatcatgtagcctagcgtggcttcgaactttctgtcccctcctgccagtgctataagcactccgggcagggctctacacactgagaactgtggatttttgttttgctttgtttggtttcatttctgggttttgctgatgttgtctcttttgaaacagggagttggtctgggactcgcaatccccctgcctcgactcctgtgagtggcctcatcacttttctggctctggtcgcggatcccaggactggggaatccactaagtcagagaggtagtctcggccagcactgaagactaagcttgctgcatcaggagggcgcatgcgctctggaaagaatgctgtttttcacctccacgtacccaccccaccctcaggcacgtatctagggaaggcggctactctttcccgaccgtcccttcaccacttccggcggaagcggcctcaacaagggaaactttattgttcccgtgcggcagcgccggcgacgatgtcggtgaattatgcagcagggttgtcgccttacgcggataagggcaagtgcgggctgcccgaggtgagagctgcagtgttcgagtcaccgaatgaccccagacggggagcggccgggcgaagcggggcattgtgggaacagcatgaaacatagagtctagacaatgggagggacttccctggaaggagggggcgggaagtgacacccactgctcaggctgggtgacacggaggaacctgggtcagggaaggagtgaggggctaatgggaacgagaccaaatcgtcaggtcagggagtggacgtcttaattgtcagggaaaacctggagctgtgggaagatctgcagggccggaagctggcaccctttcctgggggcatccctgtaccccactctgtactcctccctgaatctgggccagatggatcctgagtgtggggctgtcagtctttgttgtttctgaaggggtgtagtagggtggagggttctctctttgtagcccaggctggccccgaactcacaggaatcttcctgcctctgcctcccgagtactggtgttaaaggcctgcgacaccatgccctgcctgcctcatgttaacggagcccctcggcatgtgcaaaggccctggggtaggacacagcgatgtgttaagagtgtggctggaactgagggatgttgagaaggggcagacagagcattgacccacaggaggcctgccacttgggaggtcacgtgttgcctcctcctgccctctgaccctacagatcttcgacccaccagaggagctggaacgcaaggtgtgggagctggcccggctaatgtggcagtcctccagcgtggttttccacacgggcgccggcatcagcaccgcctctggcatccccgacttcaggtcggtgaacaatgaggggaaactgaggcagggctcttgctgatacacctctagggactaaggtgaggtggcagaaggacctttcagttttcctatgtctctgaaaggcccagcaaaaactccacttgcccaaggatcagagcatgcccacataatgtagtagctgcaaggcatgctgggaaatgtattcatgcagctcggccagccactgggttttctagggtcagaatgactcaatgttgtggagtgactagctcacccataaccttcggtggcagcctatgaccactgtaaagcattctgggaacccgcagccggtcttaaccagtactcagccccagctccactagctcccctgcctttgtttacccccagaccctcagcccctccgcctctttcatgaagacgtcccatgctctttagaattcttgcctacccggcagtcattgtctccaccacaggatacagaaccaccctaagaacggggctggggccagcatgggctgacttcagcattaacaggaagagcccacagcctggaaaatgaatgtttgacctctgtgcctgtcctcctctgcttgctatcctcagaggcccccatggcgtgtggaccatggaggaacgcggcctggcccccaagtttgacaccaccttcgagaatgctcggccctcgaagacccacatggccctggttcagctagaacgcatgggcttcctcagcttcctggtcagccagaacgtagacgggctgcacgtgcgctcgggcttccccaggtccgatgagtcggccggggggctggggctcaattgcatctcttcctccttattggaccagggtgccctagctcaggggcatttattagggatgcaaagagggggaaggctcggagcccaaagccgggcagtacttggtcaaattggggggatgtaggcagggttccacccctcactggggtggggtggggaggttggcaggggctttagggccaactgactgttgcggcagagggcgagtacatgacagaatttctccatgtcttggctaccctgggacttgccctgtagatcaggctcacagagatccacttgcctctgcctcccaatggagggattaaaggggtgtgccgcggctagccaacaggttttttgtagtggggaggaggtatttgtgagtttgtgaagacaccgtcagacagctatgcatgcaacagttcctcagaatgggacaggctgggacatcctccaggaaagagaagtggacgccccttgtgagtgagccccctaggaaggctttctggaggaggaggcagactgagcagctggtccaagccccaggatctgggaagggtccttactggcatcttcttctgcagggacaagctggcagagctgcacggaaacatgtttgtagaggaatgtcccaagtgtaagacgtgagtgttgcccgtgtaacacaagaccctgctcctcccctaagccagctcagggtcctagcatggcctagagaaggccagagacagtcagatgaaaacagaccaacaaaggctcgtagacaaatggggcaaaccctttcttcacagccccccaagggagaagctggtcatgcccttggcaatgaggggttctccctgccctgcctttgctatggctctgggctgggtgtcaacatggcttgcacaccagagcctcggggtgttgagcaggactgtgcctcctgagctttggcccctgactccctcttaggggttctttattcagacaggacttcattaaagttgcccaggttatcctgaaatttggcatcctcctgcctcagcttcctgcgtgctgagctcacaggtgcgagccatcatgtttcccttagcctcctccctcccgcaccctcccttgtgtggatgccagcggctgacactggctcagccccctctgcttgtgttttgaggcagacttgctctcagaacctgtacctctttggtttagctgagtggccagtgaactcctggggtccccagctcagcctcccaagctctggggcgacaatcctgctccaccatgctcttcgttttccatggatgactcaaacacagagcccggagtctgcttgccaggcactttcagactgagtagtctgtctgtctatccctcggttgtttgtcctgctcagtagcccatgggttgagcctggctacaccaccccccagctgagagaacattagaaaatgtgtgtgcacccttccccctttacaggcagtacgtcagagacacggttgtgggcaccatgggcctcaaggccacaggccggctctgcaccgtggccaagaccaggggacttcgggcctgtaggtaggtgactttcctcacaccctccacctgtgaggaggtgggcgccccctgactccattccctgactgccacagaggggagctgagagacaccattctggactgggaggactcgttgcctgaccgggacctgatgctcgctgatgaggccagcaggtgggaccccacactccccaggtggaaatgggtggggatggctacgcagacctccacagaaggtcgaaactcgggtgggacagcagggttgtccccatcacccccagcccagggccctgggcaccataggtgcagaggagaagctgagacacagagtgagtggaaggtttcttcgcgttcctgctttcaggtttttgtggtagtggggagaatcctgggcctgtgtgcactaaacaagccctctgcagaatcagccccttcgctggaggattctaggcaagagccccaccctgaccacgcccccatacctaactgggaactttaggtgggggcttcgtcctgaccacgcccccagtacctaactggggcattctaggtggggctccgcccagaccacgccccccagtacctaactggggcattctaggtggggctccaccctgacctccagctttgagtcatttcaccatacaggaatatttgctttccttttttgagagaaggtctgctgtgtagaccagactggccttagactcaagaggtctgtctaccttctgaatgctggaattcaatgcgaatgcagttcgctcaaagcttgaaccttttgatcctcctgcctcagcctgtggaatagctaggacaacaggcctgtgccacccagcctggttgtcgttaatttttaaaaaactggccgggtggtggtggcgcatgcctttaatcccagcacttgggaggcagaggcaagcagagttctgagttctaaatttcgaggccagcctggtctacagagtgagttctaggacagccaggactatacagagaaaccctgttaccctggctgtcctggaactcactttgtagaccaggctggcctcgaactccagaatccgcctgtctcggagaaaccctgtcttgaaaaaacaaaacccaaaactggacagtggtttacagtgagacaccaagactgaatctcagctcccaagtgtttctccagtgcctggtgggtgggcgtgagggctggaacctgaggtgggcctgggccccaccccaatttcctgctccctgttccaggaccgcagacctgtctgtcaccctgggtacctcgctgcagatccgccccagtgggaacctgccccttgccactaagcgccgaggaggccgtctggtcattgtcaacctgcaacccacaaaacatgtatgtaccagtgccctgcccggtgccccggtgccccctagcccacgctgactgtccctcccttggcaggaccgccaggctgacctgcgcatccacggctacgtggatgaggtgatgtgcagactcatgaagcatctggggctggagattccagcctgggatggaccctgcgtgctagacaaagccctgccacctctgcctcgcccagtagcactcaaggctgagccccccgtgcatctcaatggtgcagtgcatgtttcgtataagtccaagcccaacagccctatactccacaggccccccaaaagagtgaagaccgaggctgcccccagctgacccgggtatgtggggcgggaggggaacttgaaggaaccacacatgttgaggtttttgtttgtttgtttgtttgtttgttttttatattctggggactgagcccaggtttgcatatctcaccctggtgtcttttcagatttttttttttttttttttttttttgagaccaggactccctagatcgctcaggctggtctggaactcactgctatagcccaggctggcctggaactagcctgcctgcctcagcctctactgtagtagggacagcgggcaggtacaccatgtccaacacagctcctttcttcaaagtctcactgtgtcccttgtccaggtccttccagggaatctcagggcatggggaatcccacacatgtggaagaggccgtgtcccagggatccacgtgcctctctaccaatcacacccgctcgtgactcaggatgtgcccggagcagcagtctcacacactccacacaccatt

(19) United States
(12) Patent Application Publication
(10) Pub. No.: US 2005/0204410 A1
(43) Pub. Date: Sep. 15, 2005

(54) SIRT6 transgenic non-human animals and assay methods

(76) Inventors: Margaret Karow, Putnam Valley, NY (US); Raul Mostoslavsky, West Roxbury, MA (US); Frederick Alt, Cambridge, MA (US)

Correspondence Address: REGENERON PHARMACEUTICALS, INC, 777 OLD SAW MILL RIVER ROAD, TARRYTOWN, NY, 10591, US

(21) Appl. No.: 11/078,113

(22) Filed: Mar. 11, 2005

Related U.S. Application Data
(60) Provisional application No. 60/553,308, filed on Mar. 15, 2004.

(51) Int. Cl. C12Q 1/68; G01N 33/53; G01N 33/567; A01K 67/00

(52) U.S. Cl. 800/8; 435/7.2; 435/6

(57) ABSTRACT

Assay methods for identifying agents capable of modulating SIRT6 activity or expression, as well as non-human transgenic animals useful for identifying agents capable of inhibiting aging-associated degenerative processes.

### CROSS-REFERENCE TO RELATED APPLICATIONS

This application claims the benefit under 35 USC § 119(e) of U.S. Provisional 60/553,308 filed 15 Mar. Feb. 2004, which application is herein specifically incorporated by reference in its entirety.

### BACKGROUND OF THE INVENTION

1. Field of the Invention

This invention is related to methods of screening for agents capable of modulating the SIRT6 protein, as well as SIRT6 knock-out non-human animals.

2. Description of Related Art

The term “silent information regulators” (SIR) was coined to describe a set of non-essential genes required in trans for the transcriptional repression of the silent mating type loci in the budding yeast S. cerevisiae (Rine et al. (1987) Genetics 116:9-22). ySir2 is unique among these factors in that it belongs to a large family of closely related proteins present both in prokaryotes and eukaryotes species (reviewed in Dutnall & Pillus (2001) Cell 105:161-164). More recently, ySir2 was shown to function as an NAD-dependent histone deacetylase to suppress recombination at the highly repetitive rDNA locus, and turns off transcription at multiple genomic loci. Sir2 has also been shown to promote longevity in yeast mother cells. Cells lacking Sir2 have a reduced replicative life span and cells with an extra copy of Sir2 display a much longer life span than wild type. The human Sir2 histone deacetylase gene family consists of seven mammalian sirtuins (SIRTs) which are NAD-dependent histone/protein deacetylases (Kyrylenko et al. (2003) Cell Mol. Life Sci. 60 (9):1990-1997). Human SIRT6 is a recently discovered member of the Sir2-like proteins (Frye (2000) Biochem. Biophys. Res. Commun. 273 (2):793-798.

SIRT1 is the most highly related to S. cerevisiae Sir2. Unlike yeast Sir2, which has no known targets aside from histones, SIRT1 possesses a large and growing list of targets, some of which, including p53 and forkhead transcription factors, modulate cellular resistance to oxidative and genotoxic stress. Additionally, SIRT1, like Sir2, has recently been shown to directly modify chromatin and silence transcription. Nevertheless, SIRT1 seems not to affect DNA recombination, and, since SIRT1 KO mice die perinatally, studies on lifespan have been precluded (Cheng et al. (2003) Proc. Natl. Acad. Sci. USA 100:10794-10799). Thus far, no information has been forthcoming regarding the functions of the four remaining SIRTs, SIRT4-SIRT7.

Genomic instability and DNA repair has been shown to play a role in mammalian aging. Different pathways are utilized in mammals to repair DNA breaks. Double-stranded breaks (DSBs) are repaired by non-homologous end joining (NHEJ) and homologous recombination (HR), while single-stranded breaks (SSBs) are repaired through nucleotide excision repair (NER) and base excision repair (BER). Mutant mice defective in several of these pathways are characterized by increased genomic instability, increased sensitivity to specific DNA damaging agents, and some of them developed aging-like syndromes.

### BRIEF SUMMARY OF THE INVENTION

In a first aspect, the invention features a method for screening for agents capable of binding a human SIRT6, or a biologically active fragment of SIRT6 comprising (a) contacting a test agent with a SIRT6 protein, or protein fragment, and (b) determining the ability of the test agent to bind SIRT6 protein or protein fragment. The human SIRT6 protein sequence and encoding cDNA sequence are shown in SEQ ID NO:1 and 2, respectively.

In a second aspect, the invention provides assay methods for identifying an agent capable of modulating human SIRT6 activity or expression. The screening methods of the invention include in vitro and in vivo assays. Agents capable of modulating SIRT6 activity include agents with increase or decrease SIRT6 activity or expression.

In one embodiment, SIRT6 enzymatic activity is measured in the presence or absence of a test agent to determine if the agent is capable of modulating SIRT6 activity. In a specific embodiment, as described in the experimental section below, SIRT6 activity may include, for example, NAD-dependent deacetylase activity, which may be measured by nicotinamide release. In a specific exemplification, a baseline SIRT6 activity can be determine by the release labeled nicotinamide in the presence of histones, and SIRT6 activity may be compared to the baseline level in the presence of a test agent. A test agent capable of increasing the enzymatic activity of SIRT6 is an activator, whereas a test agent capable of decreasing activity is an inhibitor.

The in vitro screening methods of the invention may be conducted in a cell-based assay system or in a cell-free assay system. More specifically, a native or recombinant human SIRT6 protein or protein fragment is contacted with a candidate compound or a control compound, and the ability of the candidate compound to increase or decrease SIRT6 activity is determined.

Similarly, the ability of an agent to modulate SIRT6 expression may be determined by quantitating SIRT6 protein in the presence or absence of the test agent. Protein synthesis can be measured in any way known to one of skill in the art, for example, incorporation of a labeled amino acid into protein and/or immunoprecipitation of SIRT6 with an antibody specific for SIRT6 protein.

In another embodiment, agents capable of modulating SIRT6 activity or expression are identified in vivo in an animal system. More specifically, a candidate agent or a control compound is administered to a suitable animal, and the effect on a SIRT6-mediated parameter is determined. For example, as described in the experimental section below, SIRT6 knock-out animals exhibit accelerated aging-related degenerative processes beginning at about 3 weeks of age. Accordingly, agents capable of modulating SIRT6 may be identified with the use of an animal such as a SIRT6 knock-out. SIRT6 knock-outs have also been determined to exhibit a decreased serum IGF-I level. Accordingly, the ability of a test agent to modulate SIRT6 may be determined by measuring serum IGF-I levels in the presence or absence of the test agent.

In specific embodiments, an agent capable of inhibiting SIRT6 activity, e.g., an SIRT6 antagonist, is an antibody. The antibody may be polyclonal or monoclonal, and may be non-human, humanized, wholly human, or chimeric. In another embodiment, the agent is capable of inhibiting SIRT6 expression. More specifically, the agent capable of inhibiting SIRT6 expression is an antisense molecule, a ribozyme or triple helix, or a short interfering RNA (siRNA) capable of silencing SIRT6 gene expression.

In an sixth aspect, the invention features a transgenic animal comprising a modification of an endogenous SIRT6 gene. As described more fully in U.S. Pat. No. 6,586,251 (herein specifically incorporated by reference in its entirety), the transgenic animal of the invention may be generated by targeting the endogenous SIRT6 gene. In one embodiment of the transgenic animal of the invention, the animal is a knock-out wherein the SIRT6 gene is altered or deleted such that the function of the endogenous SIRT6 protein is reduced or ablated. In another embodiment, the transgenic animal is a knock-in animal modified to comprise an exogenous gene. In a more specific embodiment of the knock-in transgenic animal of the invention, the transgene is a human SIRT6 gene. Such transgenic animals are useful, for example, in identifying immunosuppressive agents capable of inhibiting the human SIRT6 protein and for identifying agents capable of inhibiting one or more aging-associated degenerative processes.

In one embodiment of the SIRT6 knock-out animal, the animal comprises a conditional allele. Conditional alleles as used herein means an allele of a gene that has been modified from its native sequence such that its expression, or lack thereof, is controlled by, or is conditional upon, another event. As a non-limiting example, flanking a gene or a portion of a gene with site-specific recombination sites such as loxP sites and then exposing the animal containing the flanked gene or portion of a gene to a recombinase that recognizes the site-specific recombination sites results in either excision or inversion of the flanked sequence, depending on the orientation of the site-specific recombination sites. This excision or inversion is conditional upon exposure to the recombinase. Other uses for genetically modified nom-human organisms, especially transgenic and knockout organisms, for example transgenic and knockout mice, are familiar to skilled artisans. See, for example, US patent application publication 2005/0003543. In specific embodiments, knock-out and conditional knockout mice can be used to screen potential therapeutics for specificity. In the knockout or conditional knockout mice, a specific compound for SIRT6 is not expected to have an effect. Thus, any phenotypes caused by the administration of the potential therapeutic must be from non-specific activity (off-target activity). This type of assay may be of great value for identifying good therapeutics when the target is part of a family of similar proteins, as is Sirt6.

Other objects and advantages will become apparent from a review of the ensuing detailed description.

### DETAILED DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety.

General Description

In the experiments described herein, knock-out mice unable to express the SIRT6 gene were generated and shown to have decreased B and T cells. This observation supports the therapeutic use of a SIRT6 inhibitor as an immunosuppressant under conditions when it is desirable to suppress the immune response, for example, for a patient undergoing bone marrow transplant. Further, the knock-out animals were found to develop acute degenerative processes at around 3 weeks of age, suggesting that these animals may be useful as an animal model for identifying agents capable of inhibiting, reducing, or ameliorating age-related pathologies.

Screening Assays

The present invention provides methods for identifying agents (e.g., candidate compounds or test compounds) that are capable of inhibiting SIRT6 activity or expression. Agents identified through the screening method of the invention are potential therapeutics for use as, e.g., inhibitors of age-related degenerative processes.

Examples of agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

In one embodiment, agents that modulate SIRT6 activity or expression are identified in a cell-based assay system. In accordance with this embodiment, cells expressing a SIRT6 protein or protein fragment having SIRT6 activity are contacted with a candidate (or a control compound), and the ability of the candidate compound to increase or decrease SIRT6 activity or expression is determined. The cell may be of prokaryotic origin (e.g., E. coli) or eukaryotic origin (e.g., yeast or mammalian). In specific embodiments, the cell is a SIRT6 expressing mammalian cell, such as, for example, a COS-7 cell, a 293 human embryonic kidney cell, a NIH 3T3 cell, or Chinese hamster ovary (CHO) cell. Further, the cells may express a SIRT6 protein or protein fragment endogenously or be genetically engineered to express a SIRT6 protein or protein fragment. In some embodiments of the binding and/or modulating assays of the invention, the compound to be tested may be labeled. The ability of the candidate compound to alter the activity of SIRT6 can be determined by methods known to those of skill in the art, for example, by flow cytometry, a scintillation assay, immunoprecipitation or western blot analysis. For example, modulators of SIRT6 activity may be identified using a biological readout in cells expressing a SIRT6 protein or protein fragment. Antagonists are identified by incubating cells or cell fragments expressing SIRT6 with test compound and measuring a biological response in these cells and in parallel cells or cell fragments not expressing SIRT6. An increased biological response in the cells or cell fragments expressing SIRT6 compared to the parallel cells or cell fragments indicates the presence of an agonist in the test sample, whereas a decreased biological response indicates an antagonist.

In more specific embodiments, detection of binding and/or inhibition of a test agent to a SIRT6 protein may be accomplished by detecting a biological response, such as, for example, measuring Ca²⁺ ion flux, cAMP, IP₃, PIP₃ and transcription of reporter genes. In a specific embodiment, SIRT6 knock-out cells rescued with SIRT6 may be used. Such cells recover resistance to IR and methyl methane sulfonate (MMS) sensitivity, and thus are useful for identifying compounds which modulate the ability of SIRT6 to rescue the SIRT6 knock-out cells.

In another embodiment, agents that inhibit SIRT6-mediated activity are identified in a cell-free assay system. In accordance with this embodiment, a SIRT6 protein or protein fragment is contacted with a test (or control) compound and the ability of the test compound to bind SIRT6 is determined. Competitive binding may also be determined in the presence of a SIRT6-binding molecule, e.g., histones. In vitro binding assays employ a mixture of components including a SIRT6 protein or protein fragment, which may be part of a fusion product with another peptide or polypeptide, e.g., a tag for detection or anchoring, and a sample suspected of containing a natural SIRT6 binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature that facilitates optimal binding. The mixture is incubated under conditions whereby the SIRT6 protein binds the test compound. Incubation periods are chosen for optimal binding but are also minimized to facilitate rapid, high-throughput screening.

After incubation, the binding between the SIRT6 protein or protein fragment and the suspected binding target is detected by any convenient way. When a separation step is useful to separate bound from unbound components, separation may be effected by, for example, precipitation or immobilization, followed by washing by, e.g., membrane filtration or gel chromatography. One of the assay components may be labeled which provides for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radioative emissions, nonradiative energy transfers, or indirectly detected with antibody conjugates.

It may be desirable to immobilize either the SIRT6 protein, or fragment, or its target molecule to facilitate separation of complexes from uncomplexed forms of one of the proteins, as well as to accommodate automation of the assay. Techniques for immobilizing proteins on matrices can be used in the drug screening assays. In one embodiment, a fusion protein is provided which adds a domain that allows the protein to be bound to a matrix. For example, glutathione-S-transferase fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of SIRT6 interacting protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a SIRT6-binding protein and a candidate compound are incubated in the interacting protein-presenting wells and the amount of complex trapped in the well can be quantitated. Methods for detecting such complexes, in addition to those described above for the GST-immobilized complexes, include immunodetection of complexes using antibodies reactive with SIRT6 protein target molecule, or which are reactive with SIRT6 protein and compete with the target molecule, as well as enzyme-linked assays which rely on detecting an enzymatic activity associated with the target molecule. IGF serum levels can be assayed using standard ELISAs such as those available from R&D.

In another embodiment, agents that modulate SIRT6 activity or expression are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the SIRT6-mediated activity is determined

Antibodies to Human SIRT6

The present invention provides for an antibody that specifically binds human SIRT6 and is useful for inhibiting SIRT6 activity. According to the invention, a SIRT6 protein, protein fragment, derivative or variant, may be used as an immunogen to generate immunospecific antibodies. Such immunogens can be isolated by any convenient means, including the methods described above. Antibodies include polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

For preparation of polyclonal or monoclonal antibodies directed toward SIRT6, any technique which provides for the production of antibody molecules by continuous cell lines in culture may be used. For example, the hybridoma technique originally developed by Kohler et al. (1975) Nature 256:495-497), as well as the trioma technique, the human B-cell hybridoma technique (Kozbor et al. (1983) Immunology Today 4:72), and the EBV-hybridoma technique to produce human monoclonal antibodies (Cole et al. (1985) in “Monoclonal Antibodies and Cancer Therapy”, Alan R. Liss, Inc. pp. 77-96) and the like are within the scope of the present invention. The monoclonal antibodies for diagnostic or therapeutic use may be human monoclonal antibodies or chimeric human-mouse (or other species) monoclonal antibodies. Human monoclonal antibodies may be made by any technique known in the art, for example, as described in U.S. Pat. No. 6,596,541. Chimeric antibody molecules may be prepared containing a mouse antigen-binding domain with human constant regions (e.g., Takeda et al. (1985) Nature 314:452).

The present invention provides for antibody molecules as well as fragments of such antibody molecules. Antibody fragments which contain the idiotype of the molecule can be generated by known techniques. For example, such fragments include, but are not limited to, the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent. Antibody molecules may be purified by known techniques including, but not limited to, immunoabsorption or immunoaffinity chromatography, chromatographic methods such as HPLC (high performance liquid chromatography), or a combination thereof.

The invention also provides for single chain Fvs. A single chain Fv (scFv) is a truncated Fab having only the V region of a heavy chain linked by a stretch of synthetic peptide to a V region of a light chain. See, for example, U.S. Pat. Nos. 5,565,332; 5,733,743; 5,837,242; 5,858,657; and 5,871,907, the specifications of which are herein incorporated by reference herein.

SIRT6 Antagonists

In addition to antibodies specific to SIRT6, the invention encompasses antagonists of SIRT6, including both direct inhibitors capable of inhibiting SIRT6 activity, as well as indirect inhibitors capable of inhibiting the SIRT6 pathway. Candidate compounds can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological compounds may be subjected to directed or random chemical modifications, such as acylation, alkylation, esterification, amidification, etc. to produce structural analogs.

Inhibitory Nucleic Acids

In addition to agents capable of inhibiting SIRT6 activity, the methods of the invention encompass inhibition of SIRT6 expression with nucleic acid molecules capable of interfering with or silencing SIRT6 gene expression. In one embodiment, SIRT6 expression is inhibited by SIRT6 antisense nucleic acid comprises at least 6 to 200 nucleotides that are antisense to a gene or cDNA encoding SIRT6 or a portion thereof. As used herein, a SIRT6 “antisense” nucleic acid refers to a nucleic acid capable of hybridizing by virtue of some sequence complementarity to a portion of an RNA (preferably mRNA) encoding SIRT6. The antisense nucleic acid may be complementary to a coding and/or noncoding region of an mRNA encoding SIRT6. The oligonucleotides can be DNA or RNA or chimeric mixtures or derivatives or modified versions thereof, can be single- or double-stranded, and can be modified at the base moiety, sugar moiety, or phosphate backbone. The oligonucleotide may include other appended groups such as peptides; agents that facilitate transport across the cell membrane (see, e.g., Letsinger et al. (1989) Proc. Natl. Acad. Sci. USA 86:6553-6556) or blood-brain barrier (see, e.g WO 89/10134,). Such antisense nucleic acids have utility as compounds that inhibit SIRT6 expression.

In another embodiment, SIRT6 may be inhibited with ribozymes or triple helix molecules which decrease SIRT6 gene expression. Ribozyme molecules designed to catalytically cleave gene mRNA transcripts encoding SIRT6 can be used to prevent translation of SIRT6 mRNA and, therefore, expression of the gene product. (See, e.g., PCT International Publication WO90/11364). Alternatively, the endogenous expression of SIRT6 can be reduced by targeting deoxyribonucleotide sequences complementary to the regulatory region of the gene (i.e., the gene promoter and/or enhancers) to form triple helical structures that prevent transcription of SIRT6 in target cells in the body (see, for example, Helene et al. (1992) Ann. N.Y. Acad. Sci., 660, 27-36).

In another embodiment, SIRT6 is inhibited by a short interfering RNA (siRNA) through RNA interference (RNAi) or post-transcriptional gene silencing (PTGS) (see, for example, Ketting et al. (2001) Genes Develop. 15:2654-2659). siRNA molecules can target homologous mRNA molecules for destruction by cleaving the mRNA molecule within the region spanned by the siRNA molecule. Accordingly, siRNAs capable of targeting and cleaving homologous SIRT6 mRNA are useful in the immunosuppression therapies described herein.

Transgenic Animals

The invention includes a knock-out or knock-in animal having a modified endogenous SIRT6 gene. The invention contemplates a transgenic animal having an exogenous SIRT6 gene generated by introduction of any SIRT6-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the SIRT6 protein to particular cells. The mouse genomic SIRT6 sequence is shown in SEQ ID NO:3.

Knock-out animals containing a modified SIRT6 gene as described herein are useful to identify function. Methods for generating knock-out or knock-in animals by homologous recombination in ES cells, including conditional knock-outs, are known to the art. Animals generated from ES cells by microinjection of ES cells into donor blastocytes to create a chimeric animal, which chimeric animal can be bred to produce an animal in which every cell contains the targeted modification. A transgenic animal can be produced by introducing nucleic acid into the male pronuclei of a fertilized oocyte, e.g., by microinjection, retroviral infection, and allowing the oocyte to develop in a pseudopregnant female foster animal. Further, random transgenic animals containing an exogenous SIRT6 gene, e.g., a human SIRT6 gene, may be useful in an in vivo context since various physiological factors that are present in vivo and that could effect ligand binding, SIRT6 activation, and signal transduction, may not be evident from in vitro cell-free or cell-based assays. Accordingly, it is useful to provide non-human transgenic animals to assay in vivo SIRT6 protein function, including ligand interaction, combination therapies, etc.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Sub-Cellular Localization of SIRT6

Chromatin Fractionation. Cell fractionation was performed as previously described (Mendez and Stillman (2000) Mol. Cell. Biol. 20: 8602-8612). Briefly, cells were resuspended in 10 mM Hepes, 1.5 mM MgCl2, 10 mM KCl, 0.34M sucrose and 10% glycerol, and following centrifugation the supernatant collected as cytoplasmic fraction. The pellet was then resuspended in 3 mM EDTA-0.2 mM EGTA buffer pH 8.0, and following centrifugation, the supernatant collected as the nucleoplasm fraction. The pellet was dissolved in Laemmli buffer and collected as the chromatin fraction.

In order to determine whether SIRT6 binds to chromatin, purified extracts were obtained from retroviral transfected HT1080 cells expressing FLAG-SIRT6. Nuclear extracts were separated into nucleoplasm and chromatin-nuclear matrix sub-fractions. SIRT6 was found to co-fractionate with histones almost exclusively within the chromatin fraction. As a comparison, the SIRT1 protein fractionates mainly with the nucleoplasm fraction, even though it possesses histone deacetylase activity (Vaquero et al. (2004) Mol. Cell 16:93-105). In order to directly test whether SIRT6 localizes to the chromatin, extracts were purified from WT thymocytes, and the fractions were analyzed with the anti-SIRT6 antibody. SIRT6 was found to fractionate with chromatin as well.

### Example 2

### Generation of SIRT6 Knock-Out Mice

In order to gain insights into the in vivo function of SIRT6, SIRT6 deficient cells were generated with gene-targeting experiments in embryonic stem (ES) cells. The targeted mouse SIRT6 genomic sequence is shown in SEQ ID NO:3.

Western and Immunostaining analysis. Western analysis was carried out as previously described (Cheng et al. (2003) supra). Antibodies: anti-mouse SIRT6, anti-DNA Polymerase b (NeoMarkers); anti-a-tubulin (Sigma). For immunostaining analysis, cells were grown in cover slips, and fixed in 1% parafolmaldehyde.

Construction of the targeting vector and generation of SIRT6 deficient mice. The SIRT6 gene was replaced with a LacZ gene introduced in-frame into the first exon of the gene by methods described in U.S. Pat. No. 6,586,251, herein specifically incorporated by reference in its entirety. Briefly, the SIRT6 KO targeting vector was constructed by replacing exons 1 to 6 with a LacZ gene inserted in frame after the first 21 bp of exon 1. Notl linearized targeting vector (30 ug) was electroporated into R1 ES cells as described (Valenzuela et al. (2003) Nat Biotechnol 21 (6): 652-9). The frequency of homologous recombination was ˜5.9%. Positives clones were confirmed by Southern blot analysis; genomic DNA was digested with Kpn1 and the membranes probe with a 500 bp fragment 5′ to Exon 1. Chimeric mice were generated by injection of targeted ES clones into C57BL6/J Blastocysts. Male chimeras were mated with 129Sv females to generate F1 heterozygous mice, which were interbred to generate homozygous KO mice. Lack of SIRT6 protein in the KO mice was confirmed by Western analysis and RT-PCR. Taking advantage of the LacZ insertion, heterozygous mice were analyzed for βGal-staining, as an indication of SIRT6 expression. Using this system, the results showed ubiquitous expression of SIRT6 in most tissues, both in adult mice and in embryos during development, confirming previous results with the endogenous protein.

### Example 3

### Phenotype Studies of SIRT6 Knock-out Mice: Development of Aging-Associated Degenerative Processes

SIRT6^{-/-} and wild type littermates were observed from birth. Knock-out mice were found to die at around 34 weeks of age, and to be smaller than their littermates. While most of the organs were normal, those of the lymphoid compartments (spleen, thymus and bone marrow) exhibited a severe defect in B and T cell development. When analyzed by FACs, the spleen (10 fold smaller in knockout than wildtype) still carry small numbers of mature B (B220+IgM+) and T cells (CD4+ and CD8+), indicating that the phenotype is leaky. The bone marrow show an almost complete lack of precursors and progenitors, both the pro-B and pre-B populations are absent, suggesting a block/defect at a very early stage of development. The thymus presents almost no double positive (DP) cells (CD4+-CD8+), and reduced numbers of double negative (DN) cells, most of them being at the DN1 stage of development, again indicating a defect very early during development for T cells as well. The other haemopoietic lineages (granulocyte/macrophage and erythroid) were largely unaffected, suggesting that the defect is, indeed, lymphoid specific.

Strikingly at around 3 weeks of age, the mice develop acute degenerative processes, including complete loss of subcutaneous fat deposits, lordokhyphosis, colitis, and a severe lymphopenia, caused by an increased in apoptosis, as indicated with Annexin V staining in the thymus. Finally, the mice failed to thrive, and die at around 28 days. These results suggest that SIRT6 may recapitulate some aging-related pathologies.

### Example 4

### Increased Sensitivity of SIRT6 Deficient Mouse Embryonic Fibroblasts

Generation of MEFs, metaphase analysis and DNA damage assays. SIRT6 deficient mouse embryonic fibroblasts (MEFs) were generated in order to test for SIRT6 function in a homogeneous population of cells. Briefly, MEFs were generated from 13.5-day-old embryos by using standard methods. For metaphase analysis, passage 1-5 MEFs (1×106) were plated and culture for 16 hr. Colcemid (Gibco/BRL KaryoMax Solution) was added to the culture (100 ng/ml), and the cells were incubated for 3 hr. before harvesting. Cells were treated with 0.4% KCl followed by fixation into 3:1 methanol:acetic acid. SKY analysis was performed as previously described (Zhou et al. (2002) Cell 109:811-821). Chromosomal aberrations were quantified using a Nikon Eclipse microscope equipped with an Applied Spectral Imaging interferometer. To assay for sensitivity to DNA damage, MEFs were plated at 5×10⁴ into 6 well plates and 12 hr later either g-irradiated, UV irradiated, or treated with H₂O₂ or MMS for 24 hr at the doses indicated. Cells were trypsinize and counted 1 week later.

Cell cycle analysis. BrdU incorporation was assayed with anti-BrdU antibodies (BD Pharmingen) according to the manufacturer's instructions. Briefly, 5×10⁵ cells were irradiated with the indicated doses, and 24 hr. later were pulsed with BrdU for 4 hr, harvested, stained with FITC-conjugated anti-BrdU antibodies and propidium iodide, and cell-cycle profiles analyzed by flow cytometry. For G2/M analysis, cells were irradiated with the indicated doses, and 1 hr. later harvested and stained with anti-phospho H3 antibodies (Upstate), a specific mitotic marker (Wei et al. (1998) Proc. Natl. Acad. Sci. USA 95:7480-4).

Retroviral Infection. Reconstitution of MEFs was performed as previously described (Cheng et al. (2003) supra). Briefly, SIRT6 and Polb cDNA were cloned into pBabe-puro. Virus was packaged in 293T cells by cotransfection with VSV-G and Gag-Pol expressing plasmids. MEFs were infected by incubation with virus and 2 ug/ml polybrene, and 48 hr later, were selected in 2.5 ug/ml puromycin. Cells were allowed to recover from selection for 48 hr. and then plated for the different experiments.

Histological Analysis. Mouse tissue was fixed in Bouin's fixative, embedded in paraffin, sectioned at 6 um, and hematoxylin/eosin staining was performed by standard methods.

Results. SIRT6 deficient MEFs were observed to grow slower than their wild-type (WT) match controls, measured both by plating efficiency and by their ability to incorporate bromodeoxyuridine (BrdU). Given that SIRT6 is bound to chromatin (see above), and based on the role of ySir2 in genomic stability and DNA repair, the role of SIRT6 in mammalian cells was investigated. For this purpose, the cells were first challenged with different treatments known to generate DNA breaks. Strikingly, SIRT6 deficient MEFs showed increased sensitivity to ionizing radiation (IR) and hydrogen peroxide (H₂O₂). In contrast, they showed the same sensitivity as WT following exposure to UV light, suggesting that only certain types of DNA repair pathways might be affected (see below). In order to confirm that the effect was due specifically to lack of SIRT6, SIRT6 deficient cells were reconstituted with a SIRT6 expressing-retroviral vector, which restored SIRT6 levels to endogenous levels. Notably, reconstituted MEFs were rescued, showing the same sensitivity to IR, and H₂O₂ as WT cells. As an independent assay to confirm these results, SIRT6 homozygous KO ES cells were generated as described above, and tested them for IR sensitivity. SIRT6 deficient ES cells showed increased sensitivity as well. All together, these results indicated that SIRT6 is required for normal repair of DNA breaks.

### Example 5

### Genomic Instability of SIRT6 Deficient Mouse Embryonic Fibroblasts

In order to directly test whether SIRT6 function in maintaining DNA integrity, metaphases from SIRT6 deficient MEFs and karyotypes were prepared and analyzed for chromosomal abnormalities. Strikingly, chromosomal aberrations were significantly higher in the KO cells when compared to the WT controls. When analyzed by DAPI, different type of anomalies, such as fragmented chromosomes, detached centromeres, and gaps were observed even as early as passage 2 cells. To confirm the DAPI results, Spectral Karyotype Analysis (SKY) was performed. Using this technique, spontaneous translocations were detected in the SIRT6 deficient cells, which accumulate with passage. When metaphases were prepared after IR treatment, the SIRT6 deficient cells were unable to repair the breaks as efficient as WT cells, explaining the increase sensitivity to DNA damage observed. These results indicate that SIRT6 plays a role in maintaining genomic stability.

### Example 6. Intact Cell-Cycle Checkpoints and DSB Repair in SIRT6 deficient cells

Genomic instability is observed as the result of either a direct defect in repairing DNA breaks, or a malfunctioning cell-cycle checkpoint. In the later case, aberrations are generated because of the inability of the cell to arrest for enough time to repair DNA breaks generated even under normal circumstances, such as DNA replication. To determine if SIRT6 functions in cell-cycle checkpoints, MEFs were pulse-labeled with BrdU, and the cells collected at different time-points in order to measure both the G1/S and the G2/M checkpoints. Both checkpoints were intact in the SIRT6 deficient cells. Different DNA repair pathways were then analyzed.

A recombination assay was conducted in which an immunoglobulin V(D)J recombination substrate was introduced in the cells together with the recombinase RAG expressing vectors (Hesse et al. (1987) Cell 49:775-783) and the ability of the cells to repair the RAG-generated breaks is then tested. In this assay, the breaks generated are repaired through non-homologous end joining (NHEJ). When SIRT6 deficient MEFs were transfected, they were able to repair the breaks as efficiently as WT cells indicating that the NHEJ pathway is intact in these cells. It should be noted that in this transient assay, the effect of chromatin cannot be tested, thus a role of SIRT6 as a chromatin modulator in NHEJ could not be ruled out. In order to test for a general DSB repair defect, pulse field gel electrophoresis (PFGE) was performed following exposure to high doses of IR. In this assay, cells were collected at define time points after the damage, and the ability to repair the breaks is tested. SIRT6 deficient cells repaired the DNA as efficient as WT cells, indicating that SIRT6 does not play a role in DNA double-stranded breaks (DSB) repair pathways.

SIRT6 deficient cells were tested to determine if they are affected in their base excision repair (BER) pathway. Alkylating agents have been shown to generate single stranded breaks (SSBs) which are repaired mostly through BER. Cell mutants in different components of the BER pathway, such as DNA Polb and PARP-1, showed increased sensitivity to one of these agents, methyl methane sulfonate (MMS). Notably, when SIRT6 deficient MEFs were exposed to MMS, they showed increased sensitivity, which was rescued upon SIRT6 reconstitution, indicating that the increased sensitivity was due specifically to lack of SIRT6. All together the above results indicate that SIRT6 might function in maintaining genome integrity through a role in the BER pathway.

1. A method for screening for agents capable of modulating SIRT6 activity, comprising:
(a) contacting a test agent with a SIRT6 protein; and
(b) determining the ability of the test agent to modulate SIRT6 activity relative to SIRT6 activity in the absence of the test agent, wherein a test agent capable of reducing SIRT6 activity is an antagonist and a test agent capable of increasing SIRT6 activity is an agonist.

2. The method of claim 1, wherein the method is in vitro or in vivo.

3. The method of claim 3, wherein the in vitro method is conducted in a cell-based assay system or in a cell-free assay system.

4. The method of claim 2, wherein the in vivo method is conducted in an animal.

5. The method of claim 1, wherein the test agent is an SIRT6 antagonist.

6. The method of claim 5, wherein the antagonist is an antibody or antibody fragment.

7. The method of claim 6, wherein the antibody or antibody fragment is polyclonal or monoclonal.

8. The method of claim 7, wherein the antibody or antibody fragment is a non-human, humanized, wholly human, or chimeric monoclonal antibody or antibody fragment.

9. The method of claim 1, wherein the agent is capable of inhibiting SIRT6 expression.

10. The method of claim 10, wherein the agent capable of inhibiting SIRT6 expression is an antisense molecule, a ribozyme or triple helix, or a short interfering RNA (siRNA) capable of silencing SIRT6 gene expression.

11. A transgenic non-human animal, comprising a modification of an endogenous SIRT6 gene.

12. The transgenic non-human animal of claim 11, wherein the modification is an alteration or deletion of the endogenous SIRT6 gene such that SIRT6 protein expression is reduced or ablated.

13. The transgenic non-human animal of claim 12, wherein the animal is a conditional knock-out.

14. The transgenic non-human animal of claim 12, wherein the non-human animal further comprises a human SIRT6 transgene.

15. The transgenic non-human animal of claim 14, wherein the animal is a conditional knock-out.
